(19) United States
(12) Patent Application Publication
(10) Pub. No.: US 2020/0323461 A1
(43) Pub. Date: Oct. 15, 2020

(54) POINT-OF-CARE SYSTEM FOR DETECTION OF THE PHYSICAL STRESS AT DIFFERENT PARTS OF BODY

(71) Applicant: INDIAN INSTITUTE OF TECHNOLOGY, GUWAHATI, Guwahati (IN)

(72) Inventors: Mitradip BHATTACHARJEE, Guwahati (IN); Sagnik MIDDYA, Guwahati (IN); Dipankar BANDYOPADHYAY, Guwahati (IN)

(21) Appl. No.: 16/757,048

(22) PCT Filed: Oct. 15, 2018
(86) PCT No.: PCT/IN2018/050662

§ 371 Date: Apr. 17, 2020

(30) Foreign Application Priority Data
Oct. 20, 2017 (IN) 201731037222

(51) Int. Cl. A61B 5/0492; G16H 40/63; G16H 50/30; A61B 5/00

(52) U.S. Cl. CPC A61B 5/0492; G16H 40/63; G16H 50/30; A61B 5/0004; A61B 2562/0209; A61B 2560/0431; A61B 2562/0285; A61B 2562/046; A61B 2562/164; A61B 2562/222; A61B 2562/028

(57) ABSTRACT

The present invention discloses a low-cost, user-friendly and portable point-of-care system for detection of physical stress at different parts of body of a human subject. The prototype comprises a sensor arrangement, a processing unit, and a power supply. The sensor arrangement consists of a flexible and soft substrate preferably made of electrically conducting layer coated polymer facilitating detection of electric field potential of a part of a living body of the human subject such as finger-tip, tip-toe, wrist, or tongue, once they come in contact with said sensor arrangement. The sensor arrangement generates an electrical signal, which can be correlated to the stress level of the body parts at a very high-precision. The electrical signal generated by the sensor arrangement is sent to the processing unit, which may be further transmitted wirelessly to a mobile android application for the display of the results. The present system is useful for the early detection of many diseases or disorders related to heart, nerves and muscles, which can be correlated with the symptom of increase in the stress at different body parts such as finger-tip, tip-toe, wrist, or tongue, among others.

### FIELD OF THE INVENTION

The present invention relates to monitoring stress levels of different body parts of a human subject. More specifically, the present invention is directed to develop a system for point-of-care detection of the stress levels of the different body parts such as finger-tip, tip-toe, wrist, or tongue, among others. The present system is particularly useful for the early detection of many diseases or disorders related to heart, nerves and muscles, which can be correlated with the symptom of increase in the stress at different body parts.

### BACKGROUND OF THE INVENTION

Monitoring health parameter in real time is one of the major challenges and necessity of the modern day human life style [Patel, S., et al., 2012. 9(1): p. 21]. Most of the peoples are busy with the compact schedule of their professional and personal lives. Hence they go through a stressful routine almost every day for a long duration. The health issues like heart, muscles, and nervous disorders have become common not only among the elderly peoples but also among the youths across the globe [Booth, F. W., C. K. Roberts, and M. J. Laye, 2012. 2(2): p. 1143-1211]. Thus, monitoring stress levels of different body parts in the sick-hurry schedule of contemporary human life is important to maintain the minimum quality of health for a longer duration and keep a track of the real-time abnormalities of human body.

Presently, the available techniques like electromyography (EMG), electrocardiography (ECG), or electroencephalography (EEG) are the most common methods for monitoring of the health parameters [Patel, S., et al., 2012. 9(1): p. 21]. However, they not only involve costly and non-portable techniques but also available in the centralized health centers. Most of these instruments need medical experts or professional to operate as well as analyze the data. Thus, a user-friendly and portable device is perhaps the need of the hour for the real-time monitoring of the stress-related parameters.

The EMG technique has been started with the knowledge of the generation of electrical signal in the human body muscles [Shair, E. F., et al., BioMed Research International, 2017. 2017: p. 3937254]. The technique has been routinely employed in diagnosing a range of diseases and disorders in heart, nerve, and muscles [Brown, R., et al., Annals of Clinical and Translational Neurology, 2014. 1(11): p. 867-883]. EMG is also useful in diagnosing the muscle disorders such as stiffness or strain [Vasseljen, O. Jr., Johansen, B. M., and Westgaard, R. H., Scand J Rehabil Med. 1995. 27(4): p. 243-252]. In this diagnosis process, the muscle having pain are constantly monitored under EMG scanner, which is considered as a trigger point [ref. U.S. Pat. No. 5,722,420A, US20120065538A1]. The trigger point is generally identified by analyzing a spontaneous EMG activity in a muscle location where the nearby muscles are EMG ‘quite’ [ref. US20120065538A1]. Once the trigger point is identified, the treatment involves a needle electrode, which is generally inserted to locate the site of muscle spasm and pain associated with it and the treatment of the movement disorder, recurrent muscular pain and muscle degeneration or degradation issues. [Ref. US 20120065538, US 20080064977, US 20120095360, US20160151626]. Prior art studies report that to detect the body potential [Ref. U.S. Pat. Nos. 5,318,039, 5,341,813] a group of muscle needs to be separated by the use of support or restrain device arrangement. [Ref. U.S. Pat. No. 5,086,779] This limits the patient's movement and thus is not very feasible to be used in public places for POCT applications. Moreover, use of multiple electrode system coupled with a computing device makes the instrument non-portable. [ref. US20160151626, U.S. Pat. Nos. 8,170,656 B2, 6,970,737 B1] In some of the EMG devices, [ref. U.S. Pat. Nos. 6,047,202, 6,259,938, 7,963,927, 8,255,045] electrodes are placed across the spine in order to detect the pattern of body electric potential [ref. U.S. Pat. No. 5,058,602] and the pattern of the potential help in determining the health condition of the patient. Few devices [ref. U.S. Pat. Nos. 7,420,472, 6,047,202, 9,585,583] also need some range of motion techniques to generate the signal corresponding to particular muscle group and the pattern of potential comes in the form of hard copy, which is further analyzed by the experts. [ref. U.S. Pat. No. 5,513,651] However, the EMG process of diagnosis involved is costly, non-portable, complex and needs medical experts.

Apart from the EMG, the ECG and EEG have also been employed in detecting the heart-wave and brain activities of a human in order to diagnose heart and brain diseases [ref. U.S. Pat. No. 8,380,296B2, US20170020434A1]. The currently available EMG and ECG devices are comprised of multiple electrodes to track the potentials of the muscles at different body parts. [ref. US20120065538 A1, US20160151626, U.S. Pat. Nos. 8,170,656 B2, 6,915,148, US20140240223 A1, US20150373019 A1, WO2015138734 A1, U.S. Pat. No. 6,970,737 B1] The electrodes in those devices are generally coupled to an external computer integrated with the signal processing circuits for accurate detection of the symptoms. Many prior art studies have reported different electrode and signal processing patterns in order to detect the electric field potentials near the muscles. [ref. U.S. Pat. Nos. 4,832,033 A, 8,565,888 B2, 7,218,963 B2, 6,233,484 B1]. Apart from the conventional measurement of electric field potential of the body parts, electrodynamic sensors have also been employed to detect the small electrical signal from human body for diagnosis purpose. [ref. U.S. Pat. Nos. 7,885,700, 8,923,956, US20060279284]. However, a low cost, portable point of care system is yet to appear in the art, which will enable the early detection of stress-related abnormalities of a human body parts by measuring its electric field potential and indicating the abnormalities associated with heart, nervous, or muscle.

### OBJECT OF THE INVENTION

It is thus the basic object of the present invention is to develop an economic, user-friendly and portable system for detecting/monitoring stress levels of different body parts of the human subject.

Another object of the present is to develop a portable point-of-care system for detecting/monitoring stress levels of different body parts of the human subject which would be adapted to facilitate early detection of many diseases or disorders related to heart, nerves and muscles, which can be correlated with the symptom of increase in the stress at the different body parts.

Another object of the present invention the present is to develop a portable point-of-care system for detecting/monitoring stress levels of different body parts of the human subject which would be adapted to measure body-electric-potential.

Another object of the present invention the present is to develop a portable point-of-care system for detecting/monitoring stress levels of different body parts of the human subject which would be adapted to communicate/display the detected/monitored stress level results to one or more remote recipient(s).

Another object of the present invention the present is to develop a portable point-of-care system for detecting/monitoring stress levels of different body parts of the human subject which would be capable of making a wireless connection to a mobile and proficient in data transfer and display the detected/monitored stress level results on the mobile through mobile phone based application.

### SUMMARY OF THE INVENTION:

Thus according to the basic aspect of the present invention, there is an electrically conductive sensor operative on body parts comprises non-invasive flexible or soft polymer substrate with patterned planer surface involving an array of micro/nano pillars coated with conductive material defining patterned electrodes adapted to sense muscle activities.

According to another aspect of the present invention, there is also provided a point-of-care system for detection of the physical stress of body parts comprising

electrically conductive sensor comprises non-invasive flexible or soft polymer substrate with patterned planer surface with conductive material defining patterned electrodes adapted to sense muscle activity and generate sensor output based on body-potential generated by said muscle activity of said body part;
processing unit having operative connection with the said electrically conductive sensor to receive the sensor output for processing and detection of the physical stress at the body part based on the measured body-potential.

In a preferred embodiment, present point-of-care system for detection of the physical stress of body parts comprises

sensor arrangement accommodated in a housing for externally attaching with the body part of a human subject and enabling disposition of said electrically conductive sensors of said sensor arrangement in direct non-invasive contact with skin of the body part for accurate measurement of body-potential generated by the muscle activity of said body part and generate equivalent sensor output;
said processing unit having operative connection with said sensor arrangement to receive the sensor output for processing and detection of the physical stress at the body part based on the measured body-potential.

According to another aspect of the present point-of-care system for detection of the physical stress at different parts of body, the sensor arrangement comprises

said electrically conductive sensor with flexible patterned electrically conducting surface to measure the body-potential upon contact with skin of the body part;
a connecting wire and plug to establish operative connection between the sensor and the processing unit.

According to another aspect of the present point-of-care system for detection of the physical stress at different parts of body, the electrically conductive sensor comprises flexible or soft polymer substrate with pre-patterned planer surface involving array of micro/nano pillars coated with conductive material defining the patterned electrodes and having groves therebetween.

According to another aspect of the present point-of-care system for detection of the physical stress at different parts of body, the planer surface of the flexible substrate adjusts according to shape of the muscle beneath the skin and the grooves in the patterned electrodes facilitates the skin to accommodate skin all around the patterned electrodes resulting an increase in the effective contact area between the skin and the electrodes.

According to another aspect of the present point-of-care system for detection of the physical stress at different parts of body, the sensor detects the body-potential through the conductive coating of the patterned electrodes upon contact with the muscle of the human body part and generates voltage signal as the sensor output.

According to another aspect of the present point-of-care system for detection of the physical stress at different parts of body, the sensor includes contact pad for transferring the sensor output to the processing unit through the connecting wire and plug.

According to another aspect of the present point-of-care system for detection of the physical stress at different parts of body, the processing unit includes

a socket for connecting the plug;
open source development board including amplifier to amplify the sensor output which corresponds to the detected body-potential in terms of the voltage signal;
computing processor preferably Arduino UNO to process the amplified voltage signal and thereby detect the physical stress at the body part;
a wireless communication module preferably Bluetooth module to transmit computing processor's output to a remote recipient including cooperative mobile application embodied in user's mobile phone for displaying the output; and
electrical passive components including resistors with fixed resistance value to calibrate according to the sensor output.

According to yet another aspect, the present point-of-care system for detection of the physical stress at different parts of body comprises battery unit integrated with the housing and operatively connected with the sensor arrangement and the processing unit to supply power to the sensor arrangement and the processing unit.

According to another aspect of the present point-of-care system for detection of the physical stress at different parts of body, the polymer substrate is made of PDMS and the conducting material to coat the micro/nano pillars includes Aluminum or RGO.

According to yet another aspect of the present point-of-care system for detection of the physical stress at different parts of body, the housing for externally

attaching with the body part is adapted to attach with finger-tip, tip-toe, wrist, or tongue for detecting the physical stress therein.

According to a further aspect of the present point-of-care system for detection of the physical stress at different parts of body, the computing processor process the voltage signal correlates it with the physical stress by

receiving the voltage signal from the sensor arrangement corresponding to the body-potential comes out of a particular muscle of the body part;
comparing the voltage signal's amplitude and frequency with respect to a reference value to detect physical stress of the body part whereby higher amplitude and frequency of the signal corresponds to a stressed condition of the muscle compared to relaxed situation;
converting the voltage to a digital signal and transfers it with the detected physical stress wirelessly to the remote recipient including cooperative mobile application embodied in user's mobile phone for real-time display of the detected physical stress and the digital signal and/or storing data associated with the same for future analysis.

According to another aspect of the present point-of-care system for detection of the physical stress at different parts of body, the computing processor diagnosis heart, nerve and muscles based on the detected physical stress and transfers the diagnosis result to the remote recipient including cooperative mobile application embodied in user's mobile phone for real-time display and/or storing for future use.

### BRIEF DESCRIPTION OF THE ACCOMPANYING DRAWINGS

FIG. 1(a)-(d) shows isometric view of preferred embodiments of the point-of-care system for detection of the physical stress at different parts of body in accordance with the present invention.

FIG. 2 shows (a) sensor arrangement associated with the present point-of-care system for detection of the physical stress and (b) associated processing unit in accordance with the present invention.

FIG. 3 shows sensor pattern associated with the sensor arrangement of present point-of-care system for detection of the physical stress in accordance with the present invention.

FIG. 4 shows processing unit circuit associated with the present point-of-care system for detection of the physical stress in accordance with the present invention.

FIG. 5 shows comparison between sensor responses of metal-electrode (ME) and patterned-electrode (PE) sensors.

FIG. 6(A)-(D) shows responses of the PE sensor for different body-parts in accordance with an embodiment of the present invention.

FIG. 7 shows the responses of the PE sensor for working conditions while the sensor is attached to wrist in accordance with an embodiment of the present invention.

FIG. 8 shows the responses of the PE sensor for index-finger after working-out for some time in accordance with an embodiment of the present invention.

FIG. 9 shows the responses of the PE sensor for index-finger at relaxed and stressed condition in accordance with an embodiment of the present invention.

### DETAILED DESCRIPTION OF THE INVENTION WITH REFERENCE TO THE ACCOMPANYING DRAWINGS

Here the detailed description of the invention will be given with reference to the accompanying drawings that represent the preferred embodiment of this invention.

As stated hereinbefore, the present invention addresses the issues of diagnosing heart, nerve and muscles by detecting the physical stress at different parts of a human body through monitoring the body potential. It well known that the body-potential generated by muscle activity of the different body part can be a possible indicator of heart, nerve and muscle health.

The disclosed point-of-care system for detection of the physical stress at different parts of the human body of the present invention basically includes a sensor arrangement, a processing unit, and a power supply. The sensor arrangement consists of a flexible sensor with a micro/nano patterned polymeric surface coated with conducting material. The sensor is connected to the processing unit, which receives signal from the sensor and sends to a mobile application after necessary processing. The power supply unit provides the required power to work properly.

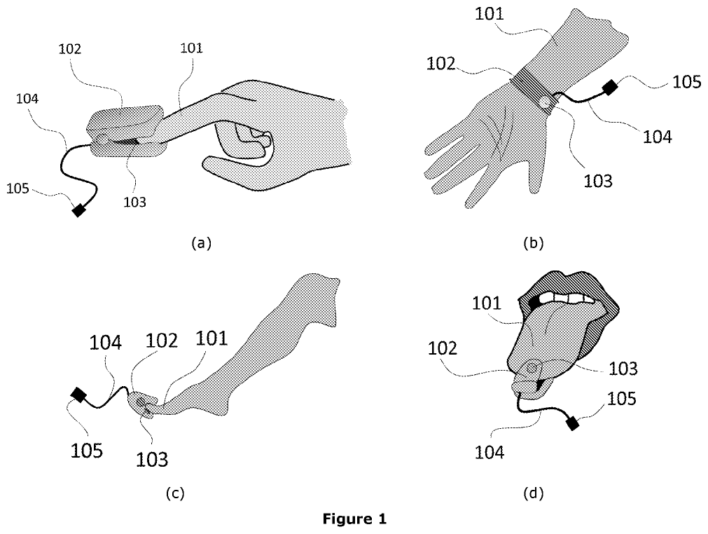

Reference is now invited from the accompanying FIG. 1 which shows the isometric view of preferred embodiments of the point-of-care system for detection of the physical stress at (a) finger tip, (b) wrist (c) leg finger and (d) tongue.

As shown in the FIG. 1, the present system includes a sensor arrangement (103) accommodated in a housing (102). The housing (102) is specifically configured to externally attach with the body part (101) as mentioned hereinbefore enabling sensor of the sensor arrangement (103) to be disposed in direct non-invasive contact with skin of the body part (101) for accurate measurement of the body-potential generated by muscle activity of said different body part. The sensor arrangement (103) also includes a connecting wire (104) and a plug (105) to establish operative connection with the processing unit.

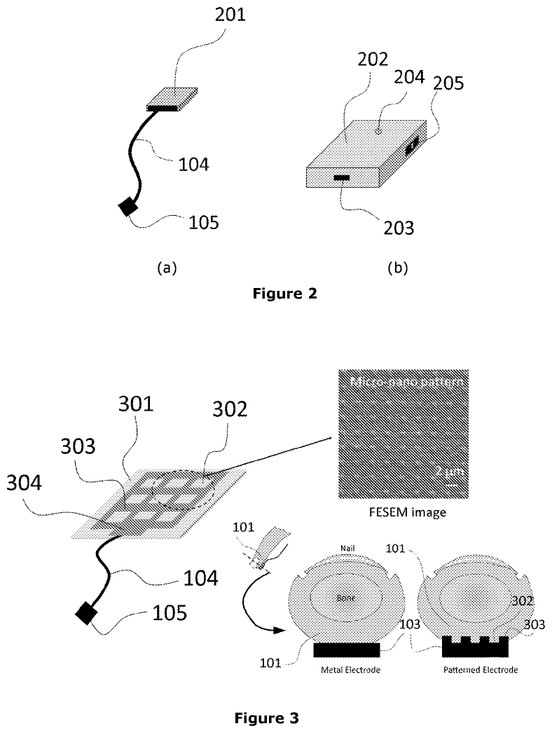

Reference is next invited from the accompanying FIG. 2 which shows (a) the sensor arrangement (103) accommodated inside the cabinet (201) with the connected wire (104) and plug (105) assembly and (b) the processing unit (202). The processing unit (202) comprises a socket (203) for connecting the plug (105). The number (204) refers to the LED indicator and the number (205) refers to the ON-OFF switch.

Reference is next invited from the accompanying FIG. 3 which shows structure of the sensor (301) of the sensor arrangement (103) for measurement of the body-potential generated by muscle activity of the different body part. As shown in the referred figure, the sensor (301) is fabricated on a flexible or soft polymer substrate preferably PDMS. The polymeric sensor substrate comprises pre-patterned planer surface with an array of micro/nano pillars coated with conductive material preferably of Al and RGO.

The micro/nano-patterns on the planner surface of the sensor (301) ensures large contact area with the skin of the body part and the flexibility/softness of the sensor (301) helps in adjusting the planer surface according to the shape of the muscle beneath the skin. The schematic illustration in FIG. 3 shows how the contact area for patterned electrodes defined by the conducting material coated micro/nano pillar on the polymeric sensor substrate increases compared to a plane metal electrode. The groves in the patterned electrodes help the soft skin to adjust in there accommodating the skin all around the patterned electrodes resulting in an increase in the effective contact area between the skin and the electrode. The number 302 and 303 show the pattern, high and low zones, respectively, made on the surface of soft material say PDMS.

The sensor (301) detects the body-potential through the conductive coating of the patterned electrodes upon touching a group of muscle of the human body part preferably index finger or wrist and generates some voltage signal as sensor output. The component 304 is the contact pad of the sensor output for connecting with the processing unit (202) through the connecting wire (104) and plug (105) and transferring the sensor output to the processing unit.

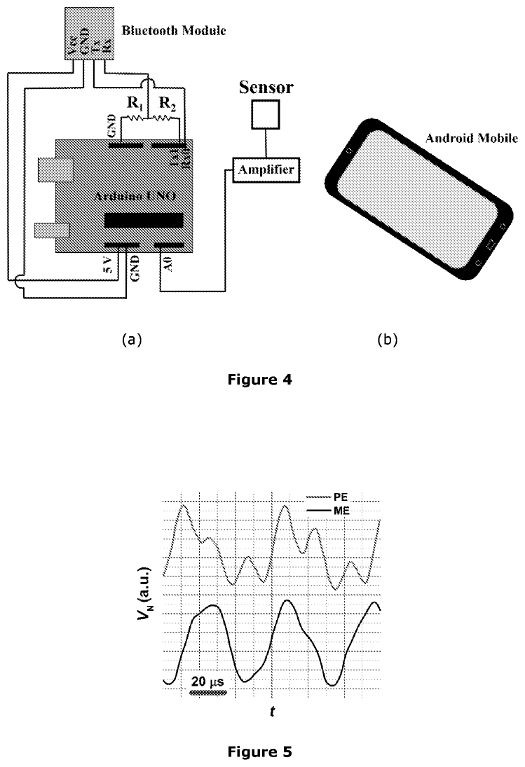

Reference is now invited from the accompanying FIG. 4 shows operating circuit of the processing unit. As shown in the figure, operating circuit comprises an open source development board, computing processor preferably Arduino UNO, a wireless communication module preferably commercial Bluetooth module (HC-06/05), and electrical passive components. The symbols R₁ and R₂ are the resistors with fixed resistance value which are calibrated according to the sensor response. The sensor output i.e. the detected body-potential in terms of voltage signal is first fed to an amplifier which amplifies the received sensor output and then transmit it to computing processor (e.g. analog input pin A0 of the Arduino UNO) for processing the amplified voltage signal to detect the physical stress at different parts of the human body and diagnosis heart, nerve and muscles based on the detected physical stress.

The computing processor after receiving the voltage signal from the sensor arrangement corresponding to the body-potential comes out of a particular muscle of the body part, compares the voltage signal's amplitude and frequency with respect to a reference value to detect physical stress of the body part whereby higher amplitude and frequency of the signal corresponds to a stressed condition of the muscle compared to relaxed situation. The computing processor also converts the voltage to a digital signal and transfers it with the detected physical stress wirelessly to a remote recipient including cooperative mobile application embodied in user's mobile phone for real-time display of the detected physical stress and the digital signal and/or storing data associated with the same for future analysis.

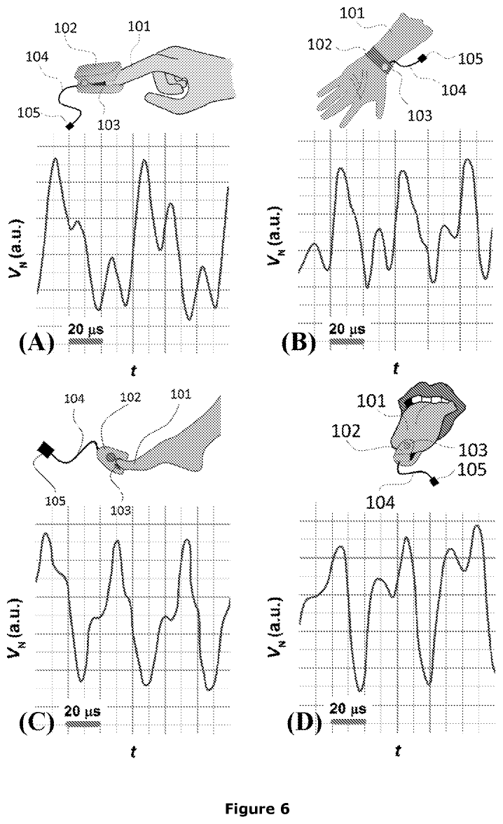

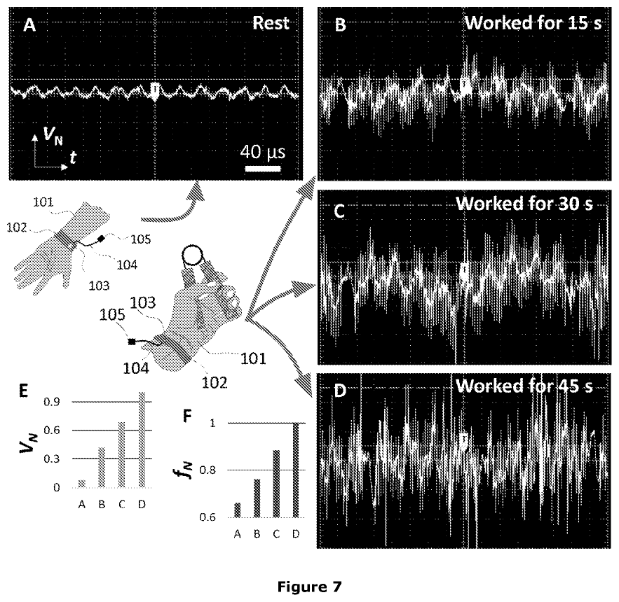

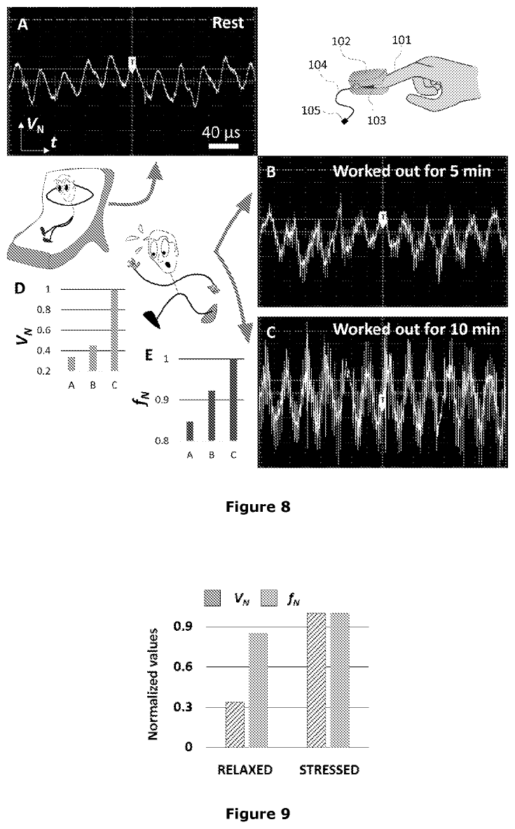

FIG. 5-8 shows the response of the sensor at different conditions. FIG. 5 shows the response of the sensor due to metal electrode (ME) and patterned-electrode (PE) attached at the index finger in rest condition. The response of the PE sensor at rest for index figure (A), wrist (B), big toe (C), and tongue (D) is shown in FIG. 6. Moreover, FIG. 7 shows the response of the PE sensor attached to the wrist at rest condition (A) and while working (B-D) for different time. Plot (E) and (F) of FIG. 7 show the change in normalized voltage (V_{N}) and frequency (f_{N}) of the body potential signal, respectively, for the cases described in images (A-D). FIG. 8 shows the response of the PE sensor attached to index finger (A) at rest condition and after working out for (B) 5 min and (C) 10 min. Plot (D) and (E) of FIG. 8 show the change in normalized voltage (V_{N}) and frequency (f_{N}) of the body potential signal, respectively for the cases described in images (A-C). Here, V_{N} refers to the normalized voltage and f_{N} stands for normalized frequency of the signal from the respective organs in arbitrary units (a.u.) and t is the time. In order to analyze the sensor response, the body potential signals were measured using a digital oscilloscope (DSO, Make—Aplab, India, Model—D37200A). This signal further can be transmitted to the mobile using wireless techniques.

FIG. 9 shows the change in normalized voltage (V_{N}) and frequency (f_{N}) of the body potential signal detected by a PE sensor attached to index finger for relaxed and stressed body condition due to work out of 10 min.

1. An electrically conductive sensor operative on body parts comprising a noninvasive flexible or soft polymer substrate with patterned planer surface involving array of micro/nano pillars coated with conductive material defining patterned electrodes adapted to sense muscle activities.

2. A point-of-care system for detection of the physical stress of body parts comprising
electrically conductive sensor comprising a non-invasive flexible or soft polymer substrate with patterned planer surface with conductive material defining patterned electrodes adapted to sense muscle activity and generate sensor output based on body-potential generated by said muscle activity of said body part; and
a processing unit having an operative connection with said electrically conductive sensor to receive the sensor output for processing and detection of the physical stress at the body part based on the measured body-potential.

3. The point-of-care system for detection of the physical stress of body parts as claimed in claim 2, further comprising
a sensor arrangement accommodated in a housing for externally attaching with the body part of a human subject and enabling disposition of said electrically conductive sensors of said sensor arrangement in direct non-invasive contact with skin of the body part for accurate measurement of body-potential generated by the muscle activity of said body part and generate equivalent sensor output;
said processing unit having an operative connection with said sensor arrangement to receive the sensor output for processing and detection of the physical stress at the body part based on the measured body-potential.

4. The point-of-care system for detection of the physical stress at different parts of body as claimed in claim 3, wherein the sensor arrangement comprises:
said electrically conductive sensor with flexible patterned electrically conducting surface to measure the body-potential upon contact with skin of the body part; and
a connecting wire and plug to establish said operative connection between the sensor and the processing unit.

5. The point-of-care system for detection of the physical stress at different parts of body as claimed in claim 4, wherein the electrically conductive sensor comprises flexible or soft polymer substrate with pre-patterned planer surface involving array of micro/nano pillars coated with conductive material defining the patterned electrodes and having groves therebetween; and
wherein the planer surface of the flexible substrate adjusts according to shape of the muscle beneath the skin and the grooves in the patterned electrodes facilitates the skin to accommodate skin all around the patterned electrodes resulting an increase in the effective contact area between the skin and the electrodes.

6. The point-of-care system for detection of the physical stress at different parts of body as claimed in claim 4,
wherein the sensor detects the body-potential through the conductive coating of the patterned electrodes upon contact with the muscle of the human body part and generates voltage signal as the sensor output; and
wherein the sensor includes a contact pad for transferring the sensor output to the processing unit through the connecting wire and plug.

7. The point-of-care system for detection of the physical stress at different parts of body as claimed in claim 2, wherein the processing unit includes
a socket for connecting the plug;
an open source development board including an amplifier to amplify the sensor output which corresponds to the detected body-potential in terms of the voltage signal;
a computing processor to process the amplified voltage signal and thereby detect the physical stress at the body part;
a wireless communication module preferably Bluetooth module to transmit computing processor's output to a remote recipient including cooperative mobile application embodied in user's mobile phone for displaying the output; and
electrical passive components including resistors with fixed resistance value to calibrate according to the sensor output.

8. The point-of-care system for detection of the physical stress at different parts of body as claimed in claim 2, comprising a battery unit integrated with the housing and operatively connected with the sensor arrangement and the processing unit to supply power to the sensor arrangement and the processing unit.

9. The point-of-care system for detection of the physical stress at different parts of body as claimed in claim 2,
wherein the polymer substrate is preferably made of PDMS and the conducting material to coat the micro/nano pillars includes Aluminum or RGO; and
wherein the housing for externally attaching with the body part is adapted to attach with finger-tip, tip-toe, wrist, or tongue for detecting the physical stress therein.

10. The point-of-care system for detection of the physical stress at different parts of body as claimed in claim 7, wherein the computing processor process the voltage signal correlates it with the physical stress by:
receiving the voltage signal from the sensor arrangement corresponding to the body-potential comes out of a particular muscle of the body part;
comparing the voltage signal's amplitude and frequency with respect to a reference value to detect physical stress of the body part whereby higher amplitude and frequency of the signal corresponds to a stressed condition of the muscle compared to relaxed situation; and
converting the voltage to a digital signal and transfers it with the detected physical stress wirelessly to the remote recipient including cooperative mobile application embodied in user's mobile phone for real-time display of the detected physical stress and the digital signal and/or storing data associated with the same for future analysis.

11. The point-of-care system for detection of the physical stress at different parts of body as claimed in claim 10, wherein the computing processor diagnosis heart, nerve and muscles based on the detected physical stress and transfers the diagnosis result to the remote recipient including cooperative mobile application embodied in user's mobile phone for real-time display and/or storing for future use.
